(19) United States
(12) Patent Application Publication
(10) Pub. No.: US 2022/0047768 A1
(43) Pub. Date: Feb. 17, 2022

(54) CONCEALED AIR SANITIZATION DEVICE WITH UV LIGHT SOURCES

(71) Applicant: Healthe, Inc., Cocoa Beach, FL (US)

(72) Inventors: Michael Alan Miller, Winter Garden, FL (US); Fredric Maxik, Cocoa Beach, FL (US); Todd Brinley Sampson, Orlando, FL (US); Ran Zhou, Rockledge, FL (US)

(73) Assignee: Healthe, Inc., Cocoa Beach, FL (US)

(21) Appl. No.: 17/404,279

(22) Filed: Aug. 17, 2021

Related U.S. Application Data
(60) Provisional application No. 63/066,614, filed on Aug. 17, 2020.

(51) Int. Cl. A61L 9/20

(52) U.S. Cl. CPC A61L 9/20; A61L 2209/134; A61L 2209/12; A61L 2209/111

(57) ABSTRACT

A concealed air sanitization device is disclosed. The concealed air sanitization device comprises a housing having an interior chamber, and an air inlet and outlet communicating between the interior chamber and an exterior environment. The concealed air sanitization device further comprises an air circulation unit configured to draw air into the interior chamber through the air inlet, one or more ultraviolet light sources disposed within the interior chamber, and one or more sensors configured to detect human presence proximate the air sanitization device. The concealed air sanitization device further comprises a processor configured to receive signals from the sensors in response to detection of human presence, and activate the air circulation unit and ultraviolet light sources in response to the detection to draw air from the exterior environment into the interior chamber and emit ultraviolet light to the air.

### CROSS-REFERENCE TO RELATED APPLICATIONS

This application claims the benefit of priority to U.S. Provisional Application No. 63/066,614 entitled “Concealed Air Sanitization Device with UV Light Sources,” filed Aug. 17, 2020, which is incorporated herein by reference in its entirety.

### TECHNICAL FIELD

The present disclosure relates generally to a concealed device configured to sanitize air using ultraviolet light. More particularly, the concealed device may be housed with a signage device that receives air through an inlet to filter the air and sanitizes the air with ultraviolet light as it passes through an interior of the device. The disclosed systems may be applied to sanitize air passing through the device when human presence is detected.

### BACKGROUND

The impact of the spread of viruses has been acutely felt throughout the world in the present time. COVID-19, SARS, and other viruses and microorganisms have had a significant and deadly impact on the way that individuals live their lives. In particular, individuals are less willing and/or able to occupy public spaces, such as malls, restaurants, theaters, public transit states, event and conference spaces, and other crowded locations, for fear of being exposed to and succumbing to a virus.

In order to combat the spread of viruses in public spaces, various precautions have been implemented. Due to the airborne nature of many pathogens including COVID-19, covering one's face with a fabric mask and maintaining physical distance from others is recommended. Additionally, proper ventilation and filtration of air within an environment may be crucial to remove pathogens from the environment. However, standard air filtration may not efficiently remove pathogens to the degree necessary to significantly impact human-to-human transmission.

More recently, ultraviolet light has been introduced as a means to sanitize surfaces and substances. The type of ultraviolet (UV) light has been classified into at least four bands depending upon the effects upon the skin of humans and other animals. Such bands include UV-A, which is defined as ultraviolet light having a wavelength in a range from 315 nm to 400 nm; UV-B, which is defined as ultraviolet light having a wavelength in a range from 280 nm to 315 nm; UV-C, which is defined as ultraviolet light having a wavelength that is in a range from 235 nm to 280 nm; and Far UV, which is defined as ultraviolet light having a wavelength that is in a range from 185 nm to 235 nm.

Ultraviolet light in the UV-C range has been used for sanitization. For example, UV light emitted at 254 nm and 265 nm has been used to destroy viruses and other microorganisms for a number of years. Far UV light (e.g., 222 nm) has been shown to have some efficacy for this use as well. However, UV light emitted in the UV-C range can have harmful impacts on humans. For example, prolonged direct exposure to UV-C light can result in eye and skin damage, such as acute corneal injury (sometimes referred to as “welder's eye”) and acute erythema. Acute effects from UV-C light include redness, ulceration or burns of the skin. Longer-term effects may include premature aging of the skin and/or skin cancer.

Still, air sanitization is often most crucial when there is human presence. Many public spaces experience waves of high traffic (e.g., public transit stations, airport terminals, and movie theaters) and thus require robust sanitization at discrete intervals where humans are present. Permanent, continuously running UV sanitization systems may have large power requirements and may thus may be obtrusive and occupy a great deal of space, which may be unsuitable for various public spaces.

As such, it would be desirable to have a concealed air sanitization system using UV light that is regulated based on the presence of humans.

### BRIEF DESCRIPTION OF THE DRAWINGS

A concealed air sanitization device is provided. The concealed air sanitization device comprises a housing, an air circulation unit, one or more ultraviolet light sources, one or more sensors, a processor, and a non-transitory, computer-readable medium. The housing comprises an interior chamber, an air inlet, and an air outlet. In some embodiments, each of the air inlet and the air outlet provide fluid communication between the interior chamber and an exterior environment. The air circulation unit is configured to draw air into the interior chamber through the air inlet. The one or more ultraviolet light sources are disposed within the interior chamber. The one or more sensors are configured to detect human presence proximate the air sanitization device. The non-transitory, computer-readable medium stores instructions that, when executed, cause the processor to receive one or more signals from the one or more sensors in response to detection of human presence, activate the air circulation unit in response to the detection to draw air from the exterior environment into the interior chamber, and activate the one or more ultraviolet light sources in response to the detection to emit ultraviolet light to the air within the interior chamber.

According to some embodiments, the one or more ultraviolet light sources are configured to emit ultraviolet light having a wavelength between about 235 nm and about 280 nm. According to additional embodiments, the one or more ultraviolet light sources are configured to emit ultraviolet light having a wavelength selected from 254 nm and 265 nm.

According to some embodiments, the one or more ultraviolet light sources are configured to emit ultraviolet light having a wavelength between about 185 nm and about 235 nm. According to additional embodiments, the one or more ultraviolet light sources are configured to emit ultraviolet light having a wavelength of about 222 nm.

According to some embodiments, the one or more ultraviolet light sources comprise one or more light emitting diodes (LEDs). According to some embodiments, the one or more ultraviolet light sources comprise one or more excimer lamps.

According to some embodiments, the concealed air sanitization device further comprises a power source in electrical connection with the processor and the one or more ultraviolet light sources.

According to some embodiments, the concealed air sanitization device further comprises one or more baffles disposed in the interior chamber. According to additional embodiments, the one or more ultraviolet light sources are disposed on the one or more baffles.

According to some embodiments, the concealed air sanitization device further comprises one or more reflective panels disposed in the interior chamber. In some embodiments, the one or more reflective panels are configured to reflect the ultraviolet light emitted from the one or more ultraviolet light sources.

According to some embodiments, the one or more reflective panels comprise one or more of aluminum and Mylar.

According to some embodiments, the concealed air sanitization device further comprises a layer of titanium oxide disposed in the interior chamber.

According to some embodiments, the concealed air sanitization device further comprises a mounting portion coupled to the housing and configured to secure the housing to one or more of a wall, a floor, and a ceiling. According to some embodiments, the concealed air sanitization device further comprises a free-standing support coupled to the housing and configured to extend upward from a floor surface to support the housing above the floor surface.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

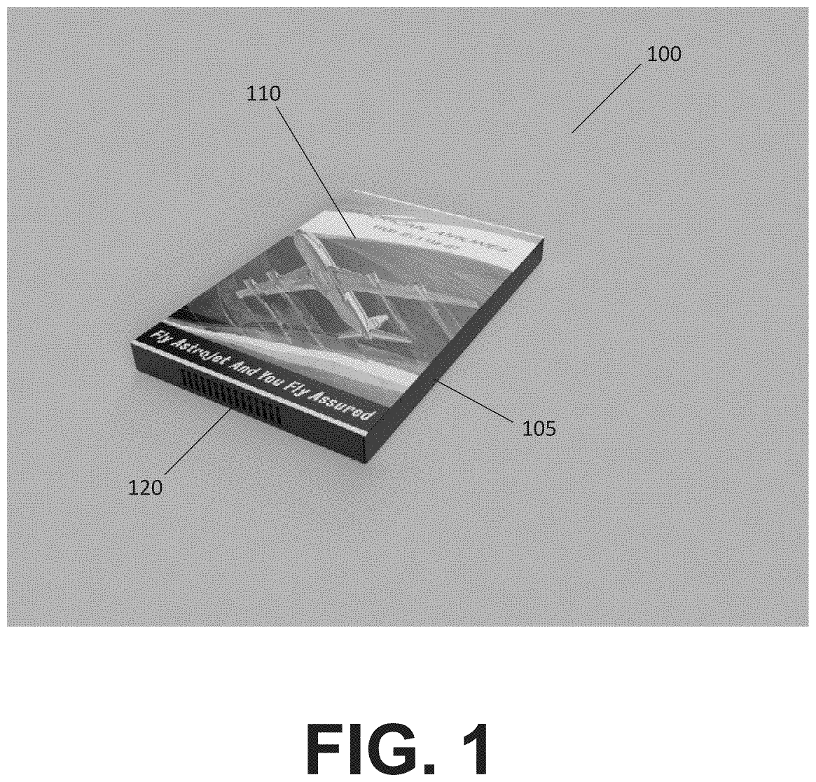

FIG. 1 depicts an illustrative concealed air sanitization device in accordance with an embodiment.

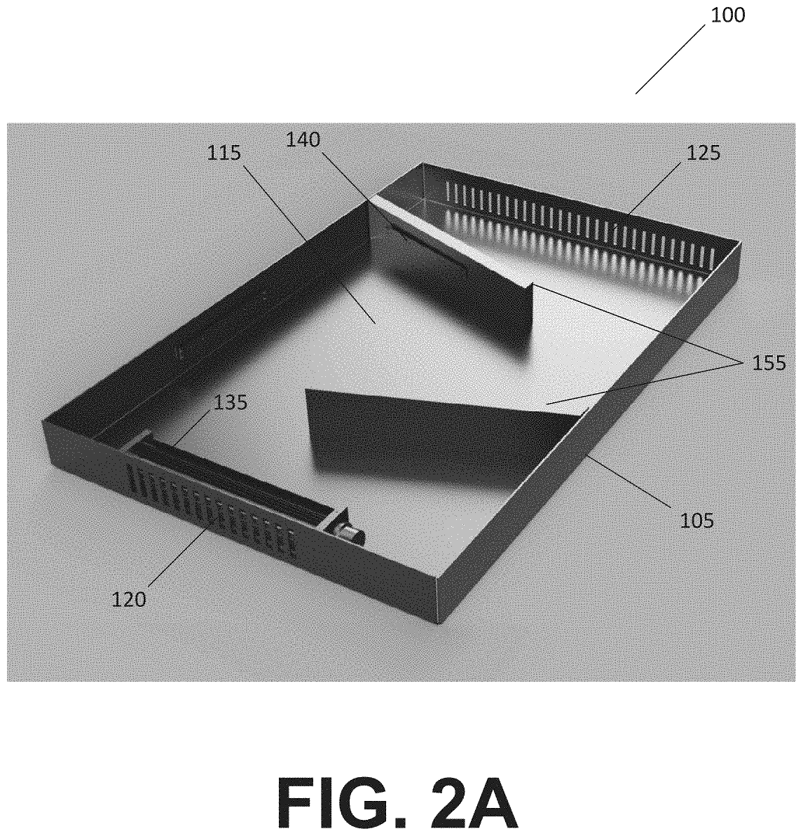

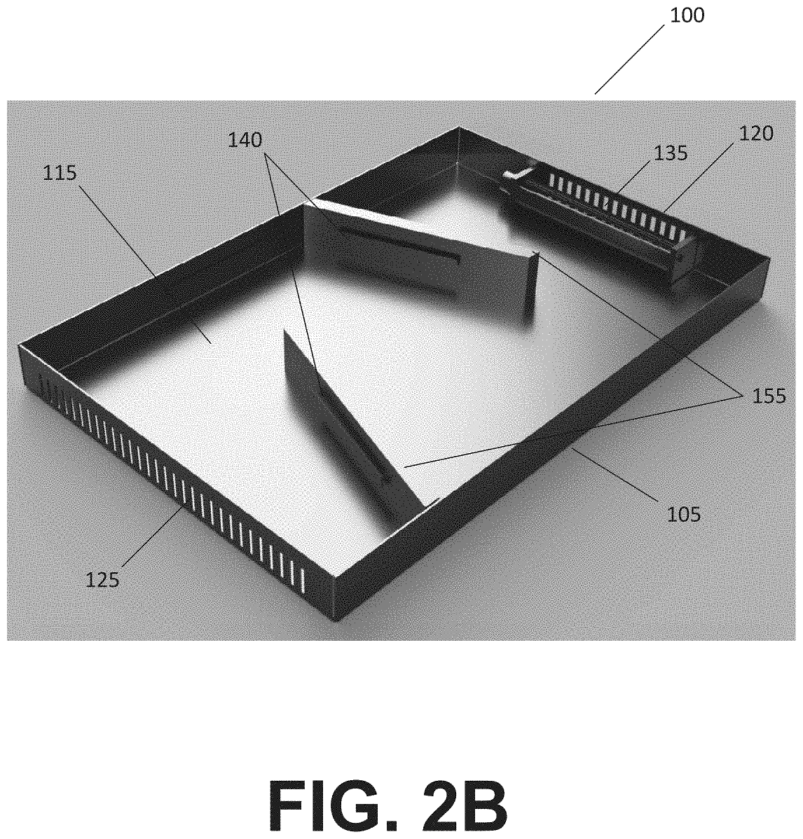

FIGS. 2A-2B depict perspective views of an interior of a concealed air sanitization device in accordance with an embodiment.

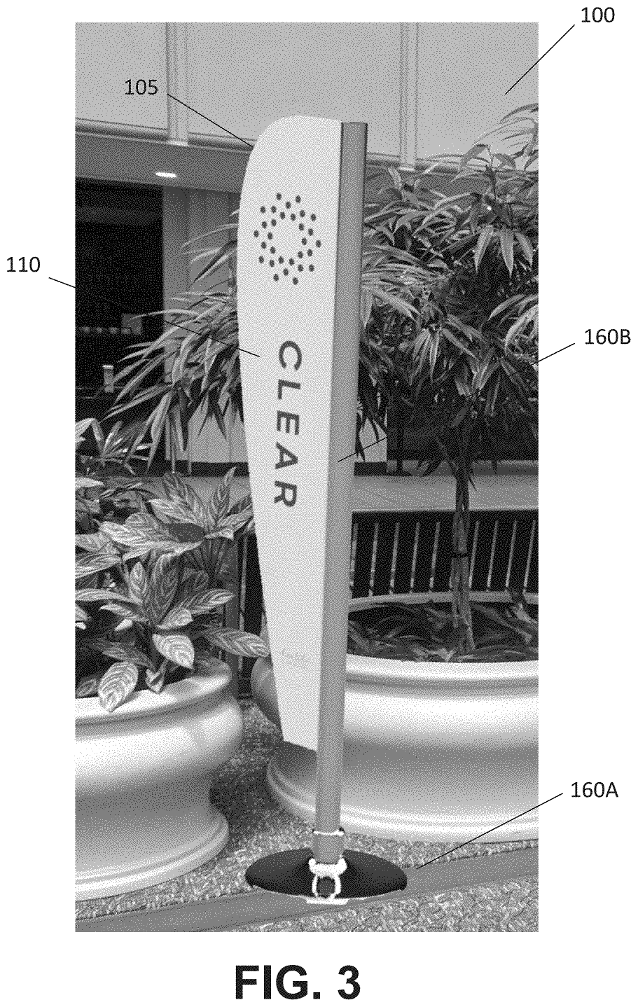

FIG. 3 depicts an illustrative concealed air sanitization device in accordance with an alternate embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope. Such aspects of the disclosure be embodied in many different forms; rather, these embodiments are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason. Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

FIG. 1 depicts an illustrative concealed air sanitization device in accordance with an embodiment. FIGS. 2A-2B depict perspective views of an interior of a concealed air sanitization device in accordance with an embodiment. FIG. 3 depicts an illustrative concealed air sanitization device in accordance with an alternate embodiment. Similar features within FIGS. 1-3 are identified with common reference numbers.

As shown in FIGS. 1-3, the concealed air sanitization device 100 comprises a housing 105, an air circulation unit (not shown), an air filter 135, one or more ultraviolet (UV) light sources 140, one or more sensors (not shown), and a control circuit (not shown).

The housing 105 may comprise an exterior surface 110 and an interior chamber 115 substantially enclosed by the exterior surface 110. In some embodiments, the housing 105 is formed in a box shape (e.g., as shown in FIGS. 1 and 2A-2B). However, the housing 105 may be formed in a variety of shapes without limitation (e.g., as shown in FIG. 3). The housing 105 may further comprise an inlet 120 and an outlet 125, where each of the inlet 120 and the outlet 125 provide fluid communication between the interior chamber 115 and an exterior environment. For example, as shown in FIGS. 1-2B, the inlet 120 and the outlet 125 may extend through the exterior surface 110 to facilitate air flow into and out of the interior chamber 115. In some embodiments, the inlet 120 and/or the outlet 125 may be formed as vents. However, additional or alternative configurations are contemplated herein as would be apparent to a person having an ordinary level of skill in the art.

In some embodiments, the exterior surface 110 or a portion thereof may comprise an informational display surface. The informational display surface may be configured for displaying indicia such as text, symbols, pictures, graphics, and logos for viewing by individuals in public or commercial spaces. Accordingly, the concealed air sanitization device 100 may serve the purpose of providing information in a public or commercial space, thereby making more efficient use of the space occupied by the concealed air sanitization device 100 and making the concealed air sanitization device 100 less conspicuous.

In some embodiments, a poster or other image comprising the indicia may be disposed on the informational display surface. However, the indicia may be directly impressed on the informational display surface by printing, stamping, etching, engraving, or other techniques as would be apparent to a person having an ordinary level of skill in the art. In some embodiments, the indicia may form the inlet 120 and/or the outlet 125. For example, the indicia may comprise one or more cutouts in the exterior surface 110 of the concealed air sanitization device 100 (e.g., cutouts in shapes forming text or a logo) that extend into the interior chamber 115, whereby air may be drawn in or expelled from the cutouts.

In some embodiments, the informational display surface may comprise a digital display screen configured to display the indicia. Accordingly, the indicia may not remain static and may be periodically updated or modified. In some embodiments, sequences or cycles of indicia may be displayed on the digital display screen according to a pattern or schedule. In some embodiments, the concealed air sanitization device may further comprise a backlight emitting visible light to illuminate the indicia on the informational display surface.

In some embodiments, the indicia comprise one or more advertisements. In some embodiments, the advertisement may comprise a picture advertisement for a business (e.g., an airline as depicted in FIG. 1) displayed on the informational display surface. In another example, the advertisement may comprise a business name or logo. In some embodiments, the indicia comprise other types of information, including but not limited to directions, instructions, reminders, news updates, or any other type of signage which may be of benefit to the general public and/or individuals present in the corresponding public or commercial space. For example, where the concealed air sanitization device 100 is situated in an airport, the informational display surface may be used to indicate directions (e.g., arrows, terminal designations, and the like), instructions (e.g., indications for boarding), and/or updates (e.g., departure or arrival times, gates, and other information). In another example, where the concealed air sanitization device 100 is situated in a mall, the informational display surface may be used to display maps, directions, and/or event information. In another example, where the concealed air sanitization device 100 is situated in a theater, the informational display surface may be used to indicate show times and/or showings associated with each room.

The air circulation unit may be configured to draw air into the interior chamber 115 through the inlet 120 and expel air out of the interior chamber 115 through the outlet 125. In some embodiments, the air circulation unit may comprise a centrifugal fan (i.e., a squirrel cage fan). However, the air circulation unit may comprise an axial flow fan, a cross-flow fan, and/or other types of fans or devices capable of creating suction to draw air through the inlet. In some embodiments, the air circulation unit is disposed proximate to the inlet 120. However, a variety of arrangements are contemplated herein.

In some embodiments, the air filter 135 may be a high-efficiency particulate absorbing (HEPA) filter. As shown in FIGS. 2A-2B, the air filter 135 may be positioned such that air drawn through the inlet 120 passes through the air filter 135 to remove dust, pollen, mold, bacteria, viruses, and/or additional airborne particles from the air. In some embodiments, the air filter 135 comprises a multi-stage air filter comprising a plurality of filter components. For example, the multi-stage air filter may include a pre-filter stage, a HEPA filter stage, an activated charcoal stage, and/or additional stages as would be known to a person having an ordinary level of skill in the art.

The one or more UV light sources 140 may be disposed in the interior chamber 115 and configured to emit UV light to the air and surfaces within the interior chamber 115 to deactivate or kill viruses, bacteria, and/or other pathogens remaining in the air. The UV light sources 140 may be disposed on any surface within the interior chamber 115 including but not limited to the top surface, the bottom surface, the sides, the baffles 155 as further discussed herein (e.g., as shown in FIGS. 2A-2B), and/or additional surfaces.

It should be understood that the one or more UV light sources 140 may provide a fluence (e.g., a combined fluence) that effectively sanitizes the air passing through the interior chamber 115 of viruses, bacteria, and/or other pathogens. The total dose of UV radiation to which air is exposed within the interior chamber 115 may be based on the fluence of the UV light sources 140 and the total exposure time, which may be controlled by the length of the flow path traversed by the air within the interior chamber 115. Accordingly, the required fluence may vary based on the length of the flow path in the interior chamber 115 as discussed herein.

In some embodiments, the UV light sources 140 are configured to emit UV light having a wavelength in the UV-C range (i.e., between about 235 nm and about 280 nm). For example, the UV light sources 140 may be configured to emit UV light having a wavelength of about 254 nm and/or about 265 nm. In some embodiments, the UV light sources 140 are configured to emit UV light having a wavelength in the Far UV range (i.e., between about 185 nm and about 235 nm). For example, the UV light sources 140 may be configured to emit UV light having a wavelength of about 222 nm. In some embodiments, a combination of UV-C and Far UV light may be emitted by the one or more UV light sources. Additional or alternate wavelengths of light that are effective in deactivating airborne pathogens are contemplated herein.

In some embodiments, the one or more UV light sources 140 comprise one or more UV light emitting diodes (LEDs). In some embodiments, the one or more UV light sources 140 comprise one or more excimer lamps. Additional or alternate types of UV light sources 140 and/or additional light sources may also be used as will be apparent to those of ordinary skill in the related art based on the teachings of this disclosure.

The one or more sensors may be configured to detect human presence proximate the concealed air sanitization device. The sensors may comprise a variety of types of sensors from which human presence can be determined and/or inferred. In some embodiments, the sensors comprise motion sensors (e.g., cameras or infrared motion sensors). In some embodiments, the sensors comprise acoustic sensors. In some embodiments, the sensors may detect changes in the environment that indicate human presence. For example, one or more infrared sensors may detect a level of carbon dioxide in a space, and human presence may be inferred therefrom.

The control circuit may comprise at least one processor and any number of additional electrical components to monitor and control the function of the concealed air sanitization device 100. In some embodiments, the processor may receive signals from the sensors and activate or deactivate components of the concealed air sanitization device 100 based on the signals. In some embodiments, it may be beneficial to selectively activate the air circulation unit and/or the UV light sources 140 when human presence or a threshold amount of human presence is detected in order to conserve power. Accordingly, the processor may activate the air circulation unit and/or the UV light sources 140 in response to a signal from the sensors indicating human presence. Further, the processor may deactivate the air circulation unit and/or the UV light sources 140 in response to a signal from the sensors indicating a lack of human presence. Alternatively, the processor may deactivate the air circulation unit and/or the UV light sources 140 where a signal from the sensors indicating human presence is not received for a specified period of time. In some embodiments, the processor may deactivate the air circulation unit and/or the UV light sources 140 after a set period of time from activation. For example, after detection of human presence, the air circulation unit and/or the UV light sources 140 may run for a time sufficient to sanitize the air in the external environment. The components may remain deactivated for a predetermined period of time and may thereafter be reactivated in response to a signal from the sensors indicating human presence.

In some embodiments, the concealed air sanitization device 100 may include a power source in electrical communication with the control circuit and the UV light sources 140. In some embodiments, the power source may include a battery. An electrical connection may be used to connect the power source to the control circuit and/or the UV light sources. For example, the electrical connection may comprise a wired connection. In some embodiments, the power source is integrated with the control circuit. In an alternate embodiment, the power source may comprise a power cord (not shown) that is integral to the concealed air sanitization device 100 and configured to connect to a remote source of power via a plug or other connector at a remote end of the cable.

As discussed herein, in some embodiments, it may be preferable to have a long flow path between the inlet 120 and the outlet 125. More particularly, it may be preferable to have a long flow path through which the air is exposed to UV light from the UV light sources 140 so that the air is sanitized for a longer period of time. Accordingly, the concealed air sanitization device 100 may further comprise one or more baffles 155 configured to redirect air within the interior chamber 115, thereby forming a longer flow path within the interior chamber than if the interior chamber had no baffles. For example, as shown in FIGS. 2A-2B, each baffle 155 may extend across the entire depth between front and back surfaces of the interior chamber 115 and may extend partially across the width of the interior chamber 115. In some embodiments, the baffles 155 may be arranged in an alternating pattern on opposing sides of the interior chamber 115 (see FIGS. 2A-2B) to form a “snaking” flow path through the interior chamber 115 between the inlet 120 and the outlet 125. Accordingly, the effective flow path of air within the interior chamber 115 may be lengthened without increasing the size of the interior chamber 115.

A “snaking” or otherwise tortuous flow path through the interior chamber may additionally be desirable to increase collisions of air particles and induce turbulent air flow within the interior chamber. Turbulent air flow may be beneficial because it decreases the efficiency (i.e., increases the length) of the path that each air particle takes through the interior chamber 115. Furthermore, a greater number of air particles may pass in close proximity to the UV light sources 140, thereby increasing a total dose of UV radiation and increasing the effectiveness in killing airborne pathogens.

In some embodiments, the interior chamber 115 may be lined or coated with reflective material to reflect UV light within the interior chamber 115, thereby increasing the exposure of air within the interior chamber 115 to the UV light. Furthermore, the reflective material may be configured to contain the UV light within the interior chamber 115 and prevent transmission out of the concealed air sanitization device 100. For example, where UV-C light is emitted by the UV light sources 140, it may be necessary to prevent transmission of the UV-C light out of the concealed air sanitization device 100 to prevent exposure to humans. In some embodiments, the reflective material comprises aluminum. In some embodiments, the reflective material comprises Mylar. In some embodiments, the reflective material comprises one or more panels disposed on the surface of the interior chamber 115. In some embodiments, the reflective material comprises a coating (e.g., a spray coating) applied to the surface of the interior chamber 115. Additional or alternate means of preventing transmission of the UV light may be utilized as would be apparent to a person having an ordinary level of skill in the art.

In some embodiments, the interior chamber 115 may be additionally lined or coated with a reactive component such as titanium dioxide (TiO₂) to induce photocatalytic effects. For example, the emitted UV light may contact the TiO₂ to induce UV-assisted TiO₂ photocatalysis (TUV), thereby producing reactive oxygen species. The reactive oxygen species may carry out redox reactions to destroy pathogens on the TiO₂ surface and/or in the air within the interior chamber 115. In some embodiments, one or more panels comprising TiO₂ may be placed on the surface of the interior chamber 115. In some embodiments, the TiO₂ may be applied as a coating (e.g., a spray coating) to the surface of the interior chamber 115. In some embodiments, the TiO₂ may be sourced from or provided in the form of anatase, ilmenite, rutile, and/or other forms. Additional or alternative reactive components may be utilized as would be apparent to a person having an ordinary level of skill in the art. For example, additional photocatalytic compounds such as zinc oxide may be used in the manner described herein.

The concealed air sanitization device 100 may be mounted to a variety of surfaces. In some embodiments, the concealed air sanitization device 100 is configured to mount to a wall. In some embodiments, the concealed air sanitization device 100 is configured to mount to a ceiling or floor. In some embodiments, the concealed air sanitization device 100 is configured to mount to a pole.

In some embodiments, the concealed air sanitization device 100 further comprises a support and is configured to be free standing. For example, as shown in FIG. 3, the concealed air sanitization device 100 may comprise support 160 including a base 160A configured to sit on a floor surface and a pole 160B to which the housing 105 is mounted, whereby the housing 105 is supported above the floor surface. In some embodiments, the support 160 may be integrated with one or more components of the concealed air sanitization device 100. In some embodiments, an interior of the support 160 may fluidly communicate with the interior chamber 115. For example, air may be drawn in and/or expelled through openings in the support 160. Accordingly, the inlet 120 and/or the outlet 125 may be located on the support 160. Furthermore, the air circulation unit, the air filter 135, the sensors, the control circuit, and/or additional components may be relocated to a portion of the support 160 as would be apparent to a person having an ordinary level of skill in the art.

The concealed air sanitization device 100 as described herein may be used in a variety of public spaces, commercial spaces, and other environments. For example, the concealed air sanitization device 100 may be particularly beneficial in spaces that occasionally need robust cleaning (e.g., due to cycles of high and low human presence). The concealed air sanitization device 100 may also be particularly beneficial in spaces where repositioning and/or removal may be frequent or foreseeable. Non-limiting examples of spaces where the concealed air sanitization device 100 may be used are airports, jet bridges, bus stops, train stations, rental car terminals, malls, restaurants, movie theaters, banks, office spaces (e.g., rental or shared office spaces), shared conference or meeting spaces, event spaces, banquet halls, amusement parks, fair grounds, and the like.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A concealed air sanitization device comprising:
a housing comprising an interior chamber, an air inlet, and an air outlet, wherein each of the air inlet and the air outlet provide fluid communication between the interior chamber and an exterior environment;
an air circulation unit configured to draw air into the interior chamber through the air inlet;
one or more ultraviolet light sources disposed within the interior chamber;
one or more sensors configured to detect human presence proximate the air sanitization device;
a processor; and
a non-transitory, computer-readable medium storing instructions that, when executed, cause the processor to:
receive one or more signals from the one or more sensors in response to detection of human presence,
activate the air circulation unit in response to the detection to draw air from the exterior environment into the interior chamber, and
activate the one or more ultraviolet light sources in response to the detection to emit ultraviolet light to the air within the interior chamber.

2. The concealed air sanitization device of claim 1, wherein the one or more ultraviolet light sources are configured to emit ultraviolet light having a wavelength between about 235 nm and about 280 nm.

3. The concealed air sanitization device of claim 1, wherein the one or more ultraviolet light sources are configured to emit ultraviolet light having a wavelength selected from 254 nm and 265 nm.

4. The concealed air sanitization device of claim 1, wherein the one or more ultraviolet light sources are configured to emit ultraviolet light having a wavelength between about 185 nm and about 235 nm.

5. The concealed air sanitization device of claim 1, wherein the one or more ultraviolet light sources are configured to emit ultraviolet light having a wavelength of about 222 nm.

6. The concealed air sanitization device of claim 1, wherein the one or more ultraviolet light sources comprise one or more light emitting diodes (LEDs).

7. The concealed air sanitization device of claim 1, wherein the one or more ultraviolet light sources comprise one or more excimer lamps.

8. The concealed air sanitization device of claim 1, further comprising a power source in electrical connection with the processor and the one or more ultraviolet light sources.

9. The concealed air sanitization device of claim 1, further comprising one or more baffles disposed in the interior chamber.

10. The concealed air sanitization device of claim 9, wherein the one or more ultraviolet light sources are disposed on the one or more baffles.

11. The concealed air sanitization device of claim 1, further comprising one or more reflective panels disposed in the interior chamber, wherein the one or more reflective panels are configured to reflect the ultraviolet light emitted from the one or more ultraviolet light sources.

12. The concealed air sanitization device of claim 1, wherein the one or more reflective panels comprise one or more of aluminum and Mylar.

13. The concealed air sanitization device of claim 1, further comprising a layer of titanium oxide disposed in the interior chamber.

14. The concealed air sanitization device of claim 1, further comprising a mounting portion coupled to the housing and configured to secure the housing to one or more of a wall, a floor, and a ceiling.

15. The concealed air sanitization device of claim 1, further comprising a free-standing support coupled to the housing and configured to extend upward from a floor surface to support the housing above the floor surface.
